(19) United States
(12) Patent Application Publication
(10) Pub. No.: US 2018/0064491 A1
(43) Pub. Date: Mar. 8, 2018

(54) TUNABLE NANOPARTICLE TAGS TO ENHANCE TISSUE RECOGNITION

(71) Applicant: The Spectranetics Corporation, Colorado Springs, CO (US)

(72) Inventors: Kevin Taylor, Colorado Springs, CO (US); Robert Splinter, Colorado Springs, CO (US)

(21) Appl. No.: 15/808,197

(22) Filed: Nov. 9, 2017

Related U.S. Application Data
(63) Continuation of application No. 11/966,214, filed on Dec. 28, 2007, now abandoned.

(51) Int. Cl. A61B 18/24; A61B 18/22; A61B 5/00; A61B 17/00

(52) U.S. Cl. CPC A61B 18/24; A61B 2017/00061; A61B 5/0059; A61B 2018/2211

(57) ABSTRACT

A method of locating and ablating a target tissue is described. The method includes providing a catheter that has at least one light guide, where the light guide is adaptable to receive light from a light source. A distal portion of the catheter is advanced through vasculature of a patient towards the target tissue. A nanoparticle dye is introduced into the patient, where the nanoparticles selectively bind to the target tissue. The target tissue is mapped by detecting fluorescence light emitted from the nanoparticle dye bound to the tissue. The distal tip of the catheter is positioned adjacent to the mapped target tissue, and a light pulse is transmitted through the light guide to ablate at least a portion of the target tissue.

### BACKGROUND OF THE INVENTION

Light-guiding catheters can be used to illuminate internal parts of the body for both diagnostic and surgical purposes. In laser assisted atherectomy, for example, the distal end of a catheter that includes one or more optical fibers inserted into the patient's vasculature and advanced to the site of a clot or occlusion in a blood vessel. Pulses of laser light travel through the optical fibers and ablate target tissue to recanalize the vessel.

A continuing challenge for laser assisted atherectomy and other types of laser surgery is distinguishing the bad tissue (e.g., the clot or occlusion tissue) from the surrounding tissue that should remain (e.g., the linings and walls of the blood vessel). One approach has been to shine light though the optical fibers, illuminating the target tissue as well as the surrounding tissue which is detected from the backscattered light traveling back through the optical fibers to a camera or some other detector. Unfortunately, it's often difficult to distinguish the occlusions from the blood vessel walls with this technique, increasing the risk that the wrong tissue will get ablated.

Another technique is to introduce dyes or contrast agents that selectively stain the target tissue at the ablation site. The dyes make it easier to distinguish the stained target tissue from the unstained surrounding tissue, reducing the risk that a vessel wall or other good tissue is inadvertently ablated. Unfortunately many types of organic dyes commonly used in in-vitro studies of tissue samples are too toxic for in-vivo use in a living patient, Additionally, many of the organic dyes and contrast agents that can be used in-vive have significant shortcomings for laser assisted atherectomy and other laser surgery procedures. For example, conventional fluorescent labels typically have short-lived fluorescent lifetimes due to rapid photobleaching after being irradiated with excitation light. This can make these labels unsuitable for the structural mapping of target tissue for the length of time required to recanalize a blood vessel using laser tissue ablation.

Conventional fluorescent labels are also sensitive to changes in environment that can effect the quantum yield of the label. For example, small changes in the pH and concentration of dissolved oxygen in the blood flowing through the vessel can cause large changes in the quantum yield and fluorescence intensity of the dyed tissue. Thus, there is a need for new dyes that allow target tissue to be distinguished from surrounding tissue that do not suffer from the high toxicity and unpredictable fluorescent properties of conventional organic dyes and labels.

### BRIEF SUMMARY OF THE INVENTION

The invention includes methods and kits for identifying and ablating target tissue in a patient, such as a chronic total occlusion. The target tissue is imaged with the help of nanoparticles that selectively bind to the tissue and fluoresce at well defined wavelengths. A dye that includes the nanoparticles may be delivered though a lumen in a light-guiding catheter whose distal end is positioned close to the tissue. When the nanoparticle dye stains the target tissue, the fluorescence light from the tissue may be transmitted though one or more light-guides (e.g., optical fibers, liquid light-guides, hollow waveguides, etc., which may be generically referred to herein as optical fibers) of the catheter to spatially map the tissue. The mapping may be used to position the distal end of the catheter, and ablation light may be transmitted through the optical fibers to ablate the tissue.

The fluorescence light may be generated by first exciting the nanoparticles with the same light used to ablate the tissue, or by using fluorescence excitation light that has a wavelength different from the ablation light. In both instances, the nanoparticles are selected to absorb fluorescence excitation light at a defined wavelength and emit fluorescence light at another defined wavelength. Since the excitation light and emitted fluorescence light have different wavelengths, there's less interference from the excitation light (e.g., the ablation light) when mapping the tissue.

Embodiments of the invention include methods of locating and ablating a target tissue. The methods may include providing a catheter having at least one light guide (e.g., optical fiber). The light guide is adaptable to receive light from a light source. The methods may also include advancing a distal portion of the catheter through vasculature of a patient towards the target tissue, and introducing a nanoparticle dye into the patient. The nanoparticle dye selectively binds to the target tissue, and the target tissue is spatially mapped by detecting fluorescence light emitted from the nanoparticle dye bound to the tissue. The methods may further include positioning the distal tip of the catheter adjacent to the mapped target tissue, and transmitting a light pulse through the light guide to ablate at least a portion of the target tissue. In some embodiments, the nanoparticle dye may be introduced by flowing the dye through a lumen in the catheter.

Embodiments of the invention may also include additional methods of locating and ablating a target tissue. The methods may include the step of providing a catheter having a plurality of light guides (e.g., optical fibers). At least a portion of the optical fibers are adaptable to receive light from an ablation light source and a fluorescence light source. The method may also include advancing a distal portion of the catheter through vasculature of a patient towards the target tissue, and introducing a nanoparticle dye into the patient. The nanoparticle dye selectively binds to the target tissue, and the target tissue is irradiated with excitation light transmitted from the fluorescence light source through the light guides. The fluorescence emitted from the nanoparticle dye bound to the tissue is detected to map the target tissue. The method may further include positioning the distal tip of the catheter adjacent to the mapped target tissue, and transmitting ablation light from the ablation light source through the light guides to ablate at least a portion of the target tissue.

Embodiments of the invention may still further include catheter and dye kits having component parts capable of being assembled to ablate target tissue in a patient. The kits may include a catheter having a plurality of light guides (e.g., optical fibers), where a proximal end of the light guides are adaptable to a light source. The kits may also include a container that holds a nanoparticle dye. The kits may still further include instructions for advancing a distal portion of the catheter through vasculature of the patient towards the target tissue, and introducing the nanoparticle dye into the patient, where the nanoparticle dye selectively binds to the target tissue. The instructions may also describe mapping the target tissue by detecting fluorescence light emitted from the nanoparticle dye bound to the tissue, positioning the distal tip of the catheter adjacent to the imaged target tissue, and transmitting a light pulse through the light guides to ablate at least a portion of the target tissue. In some embodiments, the catheter may include a lumen through which the nanoparticle dye flows into the vasculature of the patient.

Embodiments may yet further include methods of using nanoparticles to guide the distal end of a catheter with respect to the layers of a vessel wall (e.g., the wall of a vein or artery). These methods may include introducing a nanoparticle containing dye into a patient's vasculature and have the nanoparticles be absorbed by the walls of a blood vessel. The nanoparticles may be designed to selectively bind to a particular layer of the vessel walls, such as the intima, media, and/or adventitia layer. The methods may also include detecting the fluorescence light emitted from the absorbed nanoparticles and guiding the catheter to target matter and/or during lead removal with the help of the detected fluorescence light. These methods may help prevent the perforation of blood vessels both during an atherectomy and lead removal.

Embodiments of the invention may yet still further include methods of injecting nanoparticles in the lining of a vessel wall (e.g., the wall of a vein or artery). The methods may include injecting the nanoparticle into the vessel wall using the tip of a light-guiding catheter, or some other catheter. The lining of the vessel wall can migrate the nanoparticles longitudinally. The nanoparticles can emit fluorescence light and become visible when source light from a laser ablation process irradiates the vessel walls and excite the nanoparticles present.

Additional embodiments and features are set forth in part in the description that follows, and in part will become apparent to those skilled in the art upon examination of the specification or may be learned by the practice of the invention. The features and advantages of the invention may be realized and attained by means of the instrumentalities, combinations, and methods described in the specification.

### BRIEF DESCRIPTION OF THE DRAWINGS

A further understanding of the nature and advantages of the present invention may be realized by reference to the remaining portions of the specification and the drawings wherein like reference numerals are used throughout the several drawings to refer to similar components. In some instances, a sublabel is associated with a reference numeral and follows a hyphen to denote one of multiple similar components. When reference is made to a reference numeral without specification to an existing sublabel, it is intended to refer to all such multiple similar components.

FIG. 1 is a flowchart with selected steps in a method of locating and ablating target tissue using a nanoparticle dye according to embodiments of the invention;

FIG. 2 is another flowchart with selected steps in a method of locating and ablating target tissue with a nanoparticle dye and dual-light source system according to embodiments of the invention;

FIG. 3 is a flowchart with selected steps in a method of dyeing and mapping portions of a patient's vasculature to position a light-guide unit for tissue ablation according to embodiments of the invention;

FIG. 4 shows a system for locating and ablating target tissue according to embodiments of the invention;

FIG. 5 shows a dual light source system for locating and ablating target tissue according to embodiments of the invention; and

FIG. 6 shows another system for locating and ablating target tissue with an exterior lumen according to embodiments of the invention.

### DETAILED DESCRIPTION OF THE INVENTION

Methods, systems and components to identify and ablate target tissue in a patient's body are described. These include the use of a nanoparticle dyes that selectively bind to the target tissue and help to optically distinguish the target from other tissue that should not be ablated.

### Exemplary Methods of Locating and Ablating Tissue

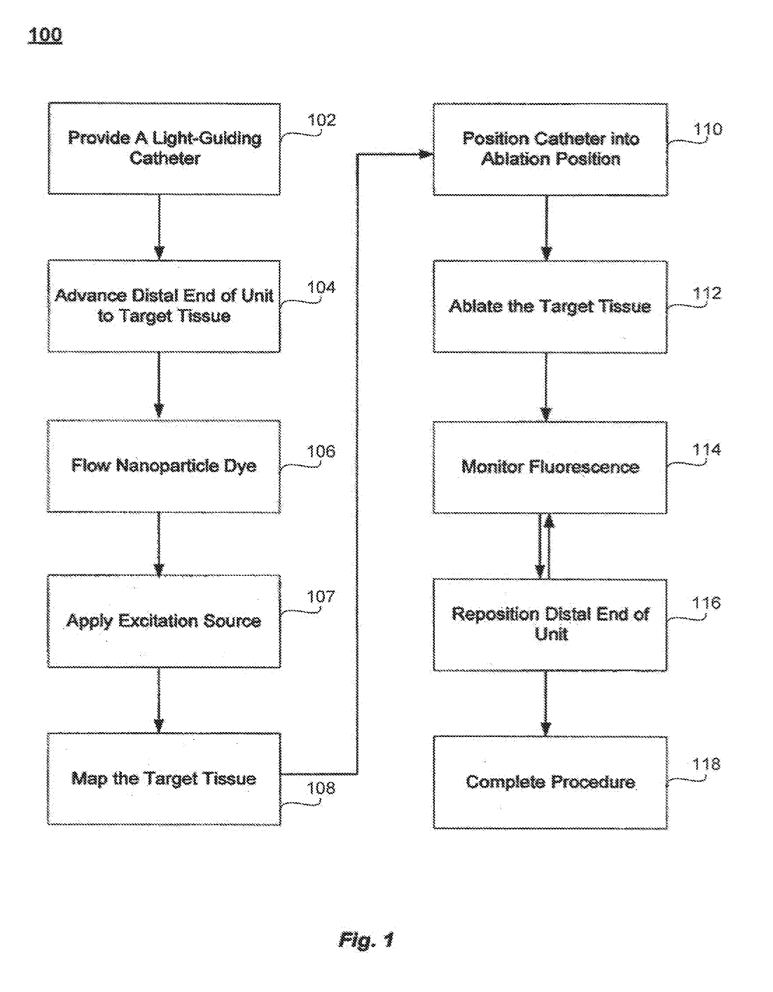

FIG. 1 is a flowchart with selected steps in a method 100 of locating and ablating target tissue using a nanoparticle dye according to embodiments of the invention. The method may include providing a catheter 102 that has at least one light guide (e.g., optical fiber). In some embodiments, the catheter may also have a lumen whose distal end is adaptable to disburse a nanoparticle dye and the light guide is adaptable to receive light from the light source. The method further includes advancing a distal portion of the catheter through vasculature of a patient towards the target tissue 104. The catheter may be advanced through the vasculature by sliding over a guidewire that is already in position proximate to the target tissue. The distal end of the guidewire may be inserted through the catheter lumen and into the patient's vasculature where it may navigate though one or more tortuous blood vessels to reach the target tissue and provide a guide path for the advancing catheter.

A nanoparticle dye may also be introduced 106 to the patient around the fluid environment of the target tissue. In examples where the catheter includes a lumen, when the distal tip of the catheter is in position proximate to the target tissue, the nanoparticle dye may flow from a dye source, through the catheter lumen, and into the fluid environment of the target tissue. The nanoparticle dye selectively binds with greater affinity to the target tissue than nearby non-targeted tissue, such a blood vessel walls. For example, if the target tissue is a calcified vascular occlusion, the nanoparticle dye may be designed to bind with greater affinity to the calcium deposits than the non-calcified vessel linings and walls.

A fluorescence excitation source may then be applied to the target tissue 107 so that the tissue is mapped 108 by a spectroscopic analysis of fluorescence light emitted from the nanoparticle dye in the tissue. The fluorescence mapping may start with exciting the nanoparticles with excitation light transmitted through the light guides to the target tissue. The excited nanoparticles then emit the fluorescence light (typically at a longer wavelength then the excitation light) which may be transmitted back through the light guides to a camera or other photodetector to structurally map the target tissue. A single wavelength (e.g., an ultraviolet wavelength from an Excimer laser) may be used to excite both the fluorescent nanoparticles and ablate the target tissue.

The imaging allows the distal tip of the catheter to be moved into an ablation position adjacent to the target tissue 110. This position may leave some space between the fiber optic tip and the tissue, or may have the tip and the tissue in physical contact. When the distal tip of the catheter is in position, an ablation light pulse may be transmitted through the light guide to ablate at least a portion of the target tissue 112. The ablation light pulse may be a laser light pulse generated by an Excimer laser. For example, the light pulse may be a 308 nm wavelength pulse generated by a XeCl Excimer laser.

Fluorescence signals from the dyed target tissue may be monitored 114 as the tissue is being ablated. When the fluorescence monitoring indicates that the distal end of the catheter is too far or otherwise in the wrong location with respect to the target tissue, the distal end may be repositioned 116 before continuing the tissue ablation. As the dual arrows in FIG. 1 show, the steps of monitoring fluorescence from the target tissue 114 and repositioning the distal end of the catheter may be repeated for a plurality of cycles (if necessary). Finally, when the target tissue has been adequately ablated (e.g., the vessel has been recanalized), the procedure is complete 118.

Additional cycles of ablation and repositioning of the catheter distal tip may also be performed. For example, following the first ablation light pulse (or pulses) that ablates a first portion of the target tissue, the dye enhanced image of the remaining target tissue may be used to reposition the light guide and/or catheter tip for a second stage of ablation. Cycles of laser ablation and catheter repositioning may be done for as many cycles as needed to disintegrate or recanalize the target tissue.

The nanoparticle dye used in the imaging of the target tissue may include doped metal oxide nanoparticles (also called “nanocrystals”) that can be designed for fluorescence excitation and emission at well defined spectral wavelengths. The nanoparticles may include metal oxide and one or more rare earth dopant elements. For example, the metal oxide may include yttrium oxide (Y₂O₃), zirconium oxide (ZrO₂), zinc oxide (ZnO), copper oxide (CuO or CuzO) gadolinium oxide (Gd₂O₃), prascodymium oxide (Pr₂O₃), lanthanum oxide (La₂O₃), and alloys of oxides. The rare earth dopant may be a metal or alloy from the Lanthanide series of elements. Specific rare earth dopants may include europium (Eu), cerium (Ce), neodymium (Nd), samarium (Sm), terbium (Tb), gadolinium (Gd), holmium (Ho), thulium (Tm), Erbium (Er), and combinations of these elements. In a specific example, the nanoparticles may include erbium doped yttrium oxide (Er³⁺:Y₂O₃).

The nanoparticles may also include one or more affinity ligands that contribute to the binding selectivity of the particles. For example, the nanoparticle dyes may include an affinity ligand that give the particles an affinity for a protein, film, mineral deposit, etc., that's found in the target tissue but not the surrounding tissue. In the case of a calcified occlusion, the affinity ligand may be a molecule that has a strong binding affinity for calcium oxide.

The composition and size of the nanoparticles depends in part on the desired excitation and fluorescence wavelengths for the nanoparticle dye. For example, different rare earth dopants can give the nanoparticles different fluorescent wavelengths, wavelength bands, and spectral profiles (e.g., fluorescent colors and the intensity distribution over the emitted wavelength spectrum).

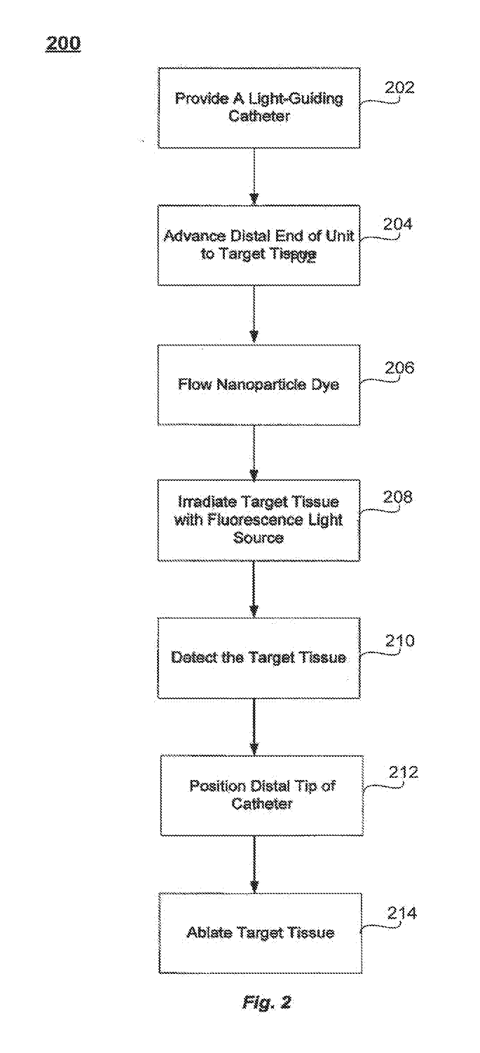

Referring now to FIG. 2, another flowchart with selected steps in a method of locating and ablating target tissue with a nanoparticle dye and dual-light source system is shown. The method 200 may include providing a catheter that has a plurality of light guides 202 (e.g., optical fibers). In some embodiments, the catheter may also include a lumen whose distal end is adaptable to disburse a nanoparticle dye. At least a portion of the light guides are adaptable to receive light from an ablation light source and a fluorescence light source.

A distal end of the catheter may be advanced through the vasculature of a patient towards the target tissue 204. The advancement of the catheter may be done by moving the catheter tip over a guidewire that has been inserted into the catheter lumen. When the distal tip of the catheter has reached a position proximate to the target tissue, the nanoparticle dye may flow through a catheter lumen 206 and selectively bind to the target tissue. The stained target tissue may then be irradiated with excitation light transmitted from the fluorescence light source through the light guides 208. The fluorescence emitted from the nanoparticles in the target tissue may be used to identify the respective components of the tissue and spatially map this composition to the target tissue 210. The distal tip of the catheter may then be positioned adjacent to the imaged target tissue 212. When the distal tip is in an ablation position, ablation light may be transmitted from the ablation light source through the light guides to ablate the target tissue 214.

In method 200, separate light sources generating light at different wavelengths are used to excite the nanoparticle dye and ablate the target tissue. For example, the fluorescent light source may generate a lower intensity and longer wavelength light than the ablation light source. The fluorescent light source may also be a wider-spectrum continuous or flash lamp while the ablation light source is a laser. The light may be transmitted down the same or different light guides from the light source to the target tissue.

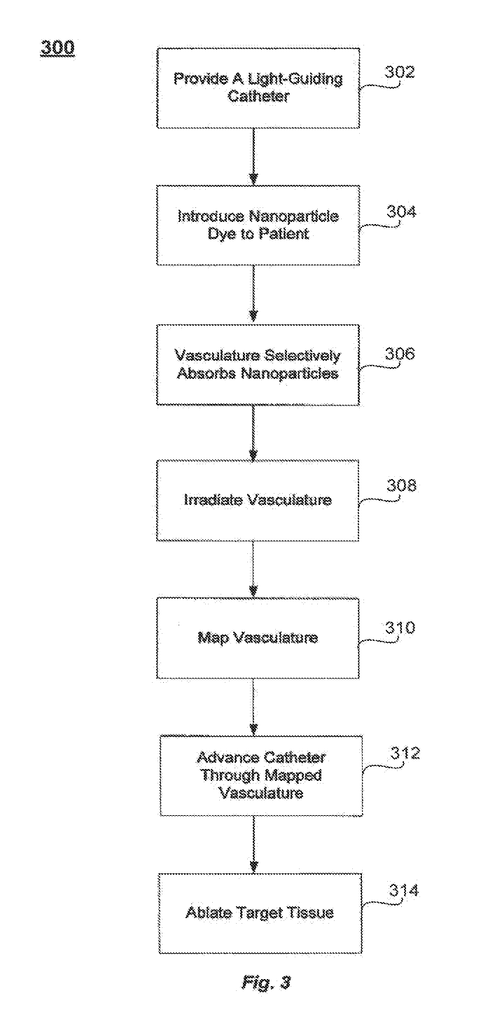

FIG. 3 shows another flowchart with steps in a method 300 of dyeing and mapping portions of a patient's vasculature to position a light-guide unit for tissue ablation according to embodiments of the invention. The method 300 may include providing a light-guiding unit 302 (e.g., a light-guiding catheter) having a distal that has a plurality of light guides (e.g., optical fibers). In some embodiments, the catheter may also include a lumen whose distal end is adaptable to disburse a nanoparticle dye, which is introduced to the patient 304. Alternatively, the nanoparticle dye may be introduced by a separate piece of equipment, such as a separate catheter or catheter tool.

The nanoparticle dye is designed to be selectively absorbed by a portion of the patient's vasculature 306. Embodiments include absorption of the dye by a selected layer or layers of a blood vessel (e.g., an artery and/or vein). For example, the nanoparticle dye may be selectively absorbed by one or more layers of a vessel wall beyond the superficial boundary where blood contacts the inner surface of the vessel. These vessel layers may include the intima, media, and/or adventitia layers, among other layers. When the nanoparticles are absorbed by the vasculature, they may immediately attach to a vessel layer, or they may migrate longitudinally along the vessel, resulting in a longer length of the vessel being dyed by the nanoparticles.

The dyed section of the vessel may be irradiated 308 with light traveling through the light guides of the light-guiding unit. The wavelength of the irradiating light is selected to excite the nanoparticles, causing them to emit fluorescence light that may be used to map the dyed vasculature 310. The mapping allows a catheter, guidewire, or other instrument to be advanced through the vasculature 312 with reduced risk of cutting or puncturing a vessel wall. When the distal tip of the light-guiding unit is advanced to the target tissue to be ablated, light pulses may be sent through the light guides to ablate the target tissue 314. The light pulses used to ablate the tissue may be the same wavelength that was used to excite the nanoparticles, or they may be a different wavelength.

### Exemplary Systems and Components

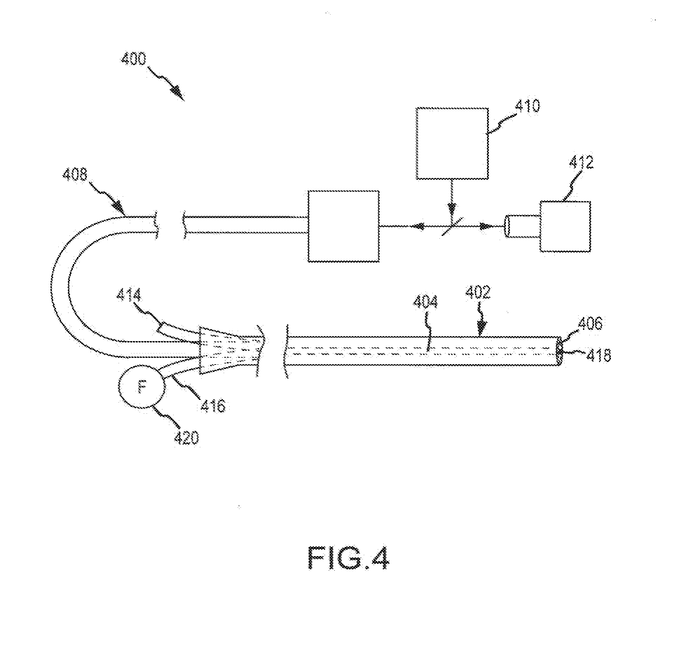

FIG. 4 shows a system 400 for locating and ablating target tissue according to embodiments of the invention. The system 400 includes a catheter 402, which has an inner lumen 404 that extends the length of the catheter. A plurality of light guides, shown here as optical fibers 406, surround the lumen 404 and may be used to transmit the ablation and imaging light. The proximal ends of the optical fibers 406 are adaptable to light conduit 408 that can transmit ablation light to the fibers from an ablation light source 410 as well as imaging light (e.g., fluorescent light) from the fibers to an imaging device 412 (e.g., a camera).

The catheter lumen 404 may also have a plurality of branched proximal ends that are adaptable to provide tools and fluid sources to the lumen. In this example, system 400 has two branched proximal ends 414 and 416. The first end 414 may be adaptable to receive a tool, such as a guidewire (not shown), that can be inserted into the catheter lumen 404 and through the lumen's distal end 418. When the guidewire is in position with respect to the target tissue, the distal end of the catheter 402 may be advanced over the guidewire towards the target tissue.

The second proximal end 416 may be adaptable to receive a nanoparticle dye solution 420 that can be pumped through the lumen 404 and into the area of the target tissue. In embodiments of the invention, the tool may be alternated with the flow of the nanoparticle dye out the lumen's distal end 418 to position and stain the target tissue during a tissue ablation procedure.

When the target tissue has been stained by the nanoparticle dye, light from the light source 410 may be used to irradiate the nanoparticles and cause them to emit fluorescence light. Some of the emitted light may be transmitted back through the optical fibers 406 and the light conduit 408 to reach an imaging device 412 (e.g., a camera). The fluorescence light normally has a longer wavelength than the ablation light used to excite the nanoparticles, so the light reacting the imaging device 412 may use a filter to block light at the ablation light wavelength (e.g., a 308 nm wavelength filter).

Embodiments of the invention include catheter and dye kits having selected component parts of a system like that shown in system 400 above. For example, the a kit may include the catheter 402 and a container with the nanoparticle dye. The kit may also include instructions for coupling the optical fibers in the catheter 402 to a light source and imaging device, and coupling the nanoparticle dye to a branch of the catheter in fluid communication with the catheter lumen 404. The instructions may further include selected steps in methods of using the system 400 to stain, image, and ablate target tissue in a patient.

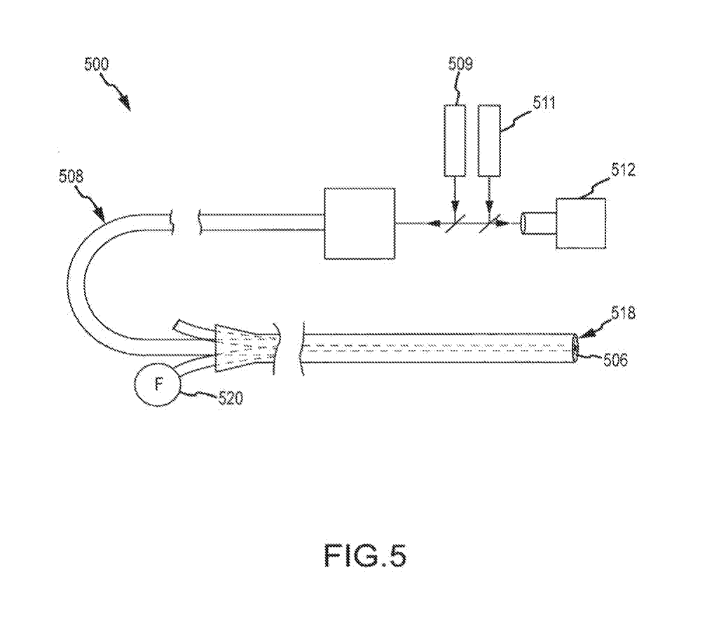

Referring now to FIG. 5, a dual light source system 500 for locating and ablating target tissue according to embodiments of the invention is shown. The system 500 includes an ablation light source 509 and a fluorescence light source 511 to generate ablation light and excitation light, respectively. The ablation light may be used to ablate target tissue proximate to the catheter's distal end 518, while the excitation light may be used to excite the nanoparticles staining the target tissue, causing the particles to fluoresce and provide an image of the target.

The two light sources 509, 511 may generate light at different wavelengths. For example, the ablation light source 509 may be an Excimer laser (e.g., a XeCl Excimer layer that generates pulses of light at 308 nm) and the fluorescence light source 511 may be a solid state laser that generates light in the near ultraviolet, visible, or infarred portions of the electromagnetic spectrum. In additional embodiments, the fluorescence light source may be a continuous lamp or flash lamp.

In some instances, a multi-wavelength light source system may have advantages over a single light source system by generating light with different wavelength and intensity characteristics. For example, the higher energy and intensity light energy bursts characteristic of a XeCl Excimer laser may be well suited for tissue ablation, but less well suited for exciting the nanoparticles. A separate fluorescence light source may provide a lower intensity, longer lasting light pulse (or even continuous light) at a wavelength that is more suitable for exciting the nanoparticles.

The dual-light source system 500 may be operated to provide excitation light to the target tissue that is asynchronous with pulsed ablation light. For example, when the catheter's distal end 518 is advanced proximate to the target tissue and the nanoparticle dye from the dye source 520 has stained the tissue. The fluorescence light source 511 may generate excitation light that is transmitted through the light conduit 508 and light guides (shown here as optical fibers 506) to reach the target tissue. The irradiated nanoparticles may then fluoresce, and some of their emitted light is transmitted back through the fibers 506 and conduit 508 for capture by the imaging device 512.

The imaged target tissue can be used to help position the distal tip 518 in an ablation position, and the ablation light source 509 may generate ablation light to ablate at least a portion of the target tissue. During the ablation step, excitation light from the fluorescence light source 511 may also be generated to image the target tissue during the ablation. Because the excitation light, fluorescence light emitted from the nanoparticles, and ablation light are all at different wavelengths, they may be transmitted through the optical fibers 506 at the same time. This can allow the target tissue to continue to be imaged during ablation.

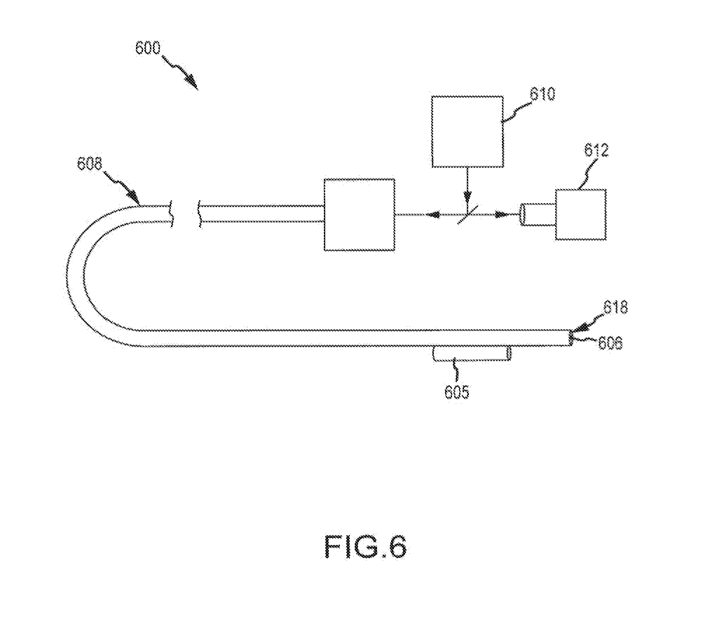

FIG. 6 shows a system 600 for locating and ablating target tissue according to embodiments of the invention. The system 600 includes a light-guiding unit that has substantially solid distal end 618 comprising a bundle of light guides (shown here as optical fibers 606), and an exterior lumen 605 through which a guidewire (not shown) may be threaded to help guide the distal end 618 to target tissue.

Light from a light source 610 may be transmitted through a light conduit 608 and the optical fibers 606 to irradiate vasculature and/or target tissue in the patient. Fluorescence light may pass back through the distal end of the optical fibers 606 and the light conduit 608 to be detected by fluorescence detector 612. The detector 612, which may include a spectrometer, may be used to extract mapping information from the dyed vasculature and/or tissue that was initially irradiated with fluorescence excitation light.

Having described several embodiments, it will be recognized by those of skill in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included.

As used herein and in the appended claims, the singular forms “a”, “an”, and “the” include plural referents unless the context clearly dictates otherwise. Thus, for example, reference to “a process” includes a plurality of such processes and reference to “the nanoparticle” includes reference to one or more nanoparticles and equivalents thereof known to those skilled in the art, and so forth.

Also, the words “comprise,” “comprising,” “include,” “including,” and “includes” when used in this specification and in the following claims are intended to specify the presence of stated features, integers, components, or steps, but they do not preclude the presence or addition of one or more other features, integers, components, steps, acts, or groups.

1-19. (canceled)

20. A catheter system and dye kit having component parts capable of being assembled to ablate target tissue in a patient, the kit comprising:
an ablation light source;
a fluorescence light source capable of emitting multiple wavelengths of fluorescence light;
a catheter comprising a plurality of light guides, wherein the catheter is coupled to the ablation light source and the fluorescence light source;
a container that holds a dye comprising nanoparticles, wherein the nanoparticles comprise one or more affinity ligands; and
instructions for:
advancing a distal portion of the catheter through vasculature of a patient towards a target tissue;
introducing the dye into the patient, wherein the dye selectively binds to the target tissue;
irradiating the nanoparticles with multiple wavelengths of fluorescence light transmitted from the fluorescence light source through the light guides and detecting the fluorescence light emitted from the nanoparticles;
mapping the target tissue by detecting multiple wavelengths of fluorescent light emitted from the nanoparticle bound to the target tissue to spatially map a composition of the vasculature;
positioning a distal tip of the catheter adjacent to the target tissue; and
transmitting a light pulse from the ablation light source through the light guides to ablate at least a portion of the target tissue.

21. The catheter system and dye kit of claim 20, wherein the dye includes nanoparticles comprising a metal oxide and a rare earth metal dopant.

22. The catheter system and dye kit of claim 21, wherein the rare earth metal comprises erbium.

23. The catheter system and dye kit of claim 20, wherein the light source is a XeCl Excimer laser.

24. The catheter system and dye kit of claim 20, wherein the light source irradiates the dye bound to the tissue to generate the fluorescence light emitted from the dye.

25. The catheter system and dye kit of claim 20, wherein a fluorescence light source, which generates light at a different wavelength than the ablation light source, irradiates the dye bound to the tissue to generate the fluorescence light emitted from the dye.

26. The catheter system and dye kit of claim 20, wherein light from the fluorescence of the dye is transmitted through the light guides of the catheter to map the target tissue.

27. The catheter system and dye kit of claim 20, wherein the catheter comprises a lumen through which the dye flows to be introduced to the patient.

28. The catheter system and dye kit of claim 20, wherein the light guides comprise optical fibers, liquid light guides, or hollow waveguides.

29. The catheter system and dye kit of claim 20, wherein irradiating the nanoparticles with multiple wavelengths of fluorescence light and transmitting a light pulse from the ablation light source through the light guides to ablate at least a portion of the target tissue are synchronized.

30. The catheter system and dye kit of claim 20, wherein irradiating the nanoparticles with multiple wavelengths of fluorescence light and transmitting a light pulse from the ablation light source through the light guides to ablate at least a portion of the target tissue are asynchronous.
